(19) United States
(12) Patent Application Publication
(10) Pub. No.: US 2010/0004605 A1
(43) Pub. Date: Jan. 7, 2010

(54) PERCUTANEOUS SAFETY NEEDLE INSERTER

(76) Inventors: Ronald B. Luther, Newport Beach, CA (US); John I. Muri, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/365,733

(22) Filed: Feb. 4, 2009

Related U.S. Application Data
(63) Continuation of application No. 11/015,758, filed on Dec. 17, 2004, now abandoned.

(51) Int. Cl. A61M 5/162

(52) U.S. Cl. 604/263

(57) ABSTRACT

An infusion device comprises an elongate housing member and a slider member. The housing member comprises a hollow cavity extending therethrough and a front plate secured to a first end of the housing. The slider member is positioned within the hollow cavity of the housing so as to be axially slidable therein. The slider member comprises a needle extending from a first end and through an aperture in the front plate. The slider is configured to slide in a direction away from the front plate to a position in which the needle is retained within the housing member. The needle is configured to be inserted into a patient to infuse fluids to the patient. The needle can be subsequently removed from the patient either before or while holding the housing member stationary and moving the slider member rearwards until the sharp tip of the needle is completely retracted within the housing.

### CROSS-REFERENCE TO RELATED APPLICATIONS

This application is a continuation of U.S. patent application Ser. No. 11/015758, filed Dec. 17, 2004, which is hereby incorporated by reference in its entirety.

### BACKGROUND OF THE INVENTION

1. Field of the Invention

The present invention relates generally to medical insertion devices, and more particularly to a percutaneous safety needle inserter.

2. Description of the Related Art

Infusion sets are commonly used in the medical field to deliver fluids and/or drugs to a patient through a subcutaneous needle or cannula. One area in which infusion sets are increasingly used is in the area of infusion pumps, often used in diabetes treatment.

Millions of people suffer from diabetes, many of whom are described as insulin-dependent. The use of insulin pumps for insulin-dependent patients has grown substantially. Insulin pumps generally deliver a constant baseline quantity of insulin to a patient throughout the day. The use of insulin pumps greatly reduces the degree of fluctuation in blood sugar levels, and generally improves the quality of life for insulin dependent diabetic patients.

Insulin pumps typically include infusion sets configured to deliver insulin from the pump, typically worn on a patient's belt, into a patient's body. The infusion sets typically include hard or soft cannulae in fluid communication with the insulin pump via a section of tubing. The cannula is generally configured to be inserted subdermally into a patient's fatty tissue (typically in a person's mid-section). Once inserted, the cannula is taped in place, and fluid is pumped through the tube, through the cannula and into a patient's tissue.

However, the cannula cannot be left inserted in the patient indefinitely, because it is a foreign object and the patient's body will eventually react to its presence. Thus, the cannula must be removed and replaced periodically (typically every one to three days, depending on the particular design). In the case of a hard cannula, the tip is generally sharp in order to allow easy insertion of the needle. Thus, once a sharp hard cannula (i.e. needle) is removed, a potential exists for a patient or a patient's caregiver to be stuck or injured by the needle. It is generally desirable to avoid such injuries for obvious reasons, including the potential for spreading of various blood-borne infections. While such needles are typically handled carefully, if the needle remains exposed after removal from the patient, the possibility for injury remains. It is therefore desirable to provide a system for increasing the safety of such infusion set needles.

### SUMMARY OF THE INVENTION

It is desirable to provide a needle insertion device configured to automatically cover a needle tip on removal of the needle from a patient. Thus, some embodiments of the present invention comprise a needle locking mechanism configured to ensure that a used and potentially contaminated needle can be completely retracted into and encased within a protective housing upon withdrawal from a patient. Covering the distal tip of a used needle helps prevent inadvertent needle pricks by an exposed, potentially contaminated needle after withdrawal from a patient.

Thus, one embodiment of the present invention provides an infusion device comprising an elongate housing member and a slider member. The housing member comprises a hollow cavity extending therethrough and a front plate secured to a first end of the housing. The slider member is positioned within the hollow cavity of the housing so as to be axially slidable therein. The slider member comprises a needle extending from a first end and through an aperture in the front plate. The slider is configured to slide in a direction away from the front plate to a position in which the needle is retained within the housing member.

Another embodiment of the present invention provides a method of preventing injury from an infusion needle. One embodiment of such a method comprises locating an infusion set comprising a slider member having a needle extending from a front end thereof into a patient, the slider member being slidably positioned within a housing member. The method is continued by removing the needle from the patient, and holding the housing member stationary while moving the slider member rearwards until a sharp tip of the needle is completely retracted within the housing. In some embodiments, the needle can be removed from the patient simultaneously with the step of moving the slider member rearwards. In some alternative embodiments, the needle can be removed from the patient before the step of moving the slider member rearwards.

In another embodiment of the invention, a safety needle inserter is provided. The safety needle inserter includes a flexible needle with a first section and a second section, and a locking mechanism configured to selectively prevent the needle from retracting into the housing when the needle is inserted into a patient. The second section of the needle is biased at an angle downward from the first section the needle. The locking mechanism may include a housing having a longitudinal groove, a slider slidably disposed within the housing, the slider having a head portion extending above a top surface of the slider. The head portion is slidably disposed within the longitudinal groove, wherein the first section of the needle is embedded in the slider. The locking mechanism also includes projections on the longitudinal groove through which the head portion may pass.

In yet another embodiment, a percutaneous safety needle inserter is provided. The safety needle inserter includes a housing having a longitudinal groove thereon, a needle having a first section and a second section, and a locking mechanism. According to one embodiment, the second section of the needle is at an angle to the first section of the needle. The locking mechanism comprises a slider attached to the needle. The slider is slidably disposed within the housing. The safety needle inserter may also include a projection protruding from a top surface of the slider, wherein the projection is slidably disposed in the longitudinal groove and the first section of the needle is attached to the slider. The longitudinal groove may have a pair of barbs through which the projection may pass in a first direction. According to one embodiment, the barbs are configured to prevent the projection from moving past the barbs in a direction opposite the first direction.

In still another embodiment, a method of injecting an intravenous fluid into a patient is provided. First, a safety needle inserter is provided. The safety needle inserter comprises a flexible, bent needle and a housing having flexible wings projecting laterally from the housing. A portion of the needle is injected into the patient and the wings are affixed to the patient after injecting. The needle is then withdrawn from the patient into a retracted position by retracting the needle completely into the housing such that a distal tip of the needle is contained within the housing. The needle is then locked in the retracted position immediately after withdrawing the needle from the patient while the wings are affixed to the patient. According to one embodiment, the housing has a longitudinal groove thereon and a slider is slidably disposed within the housing, the slider having a head portion extending from a top surface of the slider. The head portion is slidably disposed within the longitudinal groove. Locking the needle may include moving the head portion in a rearward direction along the longitudinal groove until the needle is fully contained within the housing. Each of the wings may have a projection on a top surface, the projection configured to engage an opening in the housing and an indent in the slider to prevent movement of the slider. The longitudinal groove may have a pair of barbs through which the head portion may pass in a rearward direction but not in a forward direction.

### BRIEF DESCRIPTION OF THE DRAWINGS

These and other aspects of the invention will be readily apparent to the skilled artisan in view of the description below, the appended claims, and from the drawings, which are intended to illustrate and not to limit the invention, and wherein:

FIG. 1 is an exploded perspective view of an embodiment of a needle inserter.

FIG. 2 is a perspective view of an embodiment of an infusion needle inserter in an in-use position.

FIG. 3 is a perspective view of the needle inserter of FIG. 1 in a locked position.

FIG. 4 is a perspective cross-sectional view of a needle inserter in a use position.

FIG. 5 is a perspective cross-sectional view of an embodiment of a needle inserter in a locked position.

FIG. 6 is a side cross-sectional view of an embodiment of a needle inserter in the use position.

FIG. 7 is a side cross-sectional view of an embodiment of a needle inserter in a locked position.

### DETAILED DESCRIPTION OF THE PREFERRED EMBODIMENTS

The following detailed description of the preferred embodiments and methods presents a description of certain specific embodiments to assist in understanding the claims. However, one may practice the present invention in a multitude of different embodiments and methods as defined and covered by the claims.

Referring more specifically to the drawings for illustrative purposes, embodiments of the present invention are generally shown in the Figures. It will be appreciated that the apparatus may vary as to configuration and as to details of the parts, and that the methods may vary as to the specific steps and sequence, without departing from the basic concepts, as disclosed herein. Although certain embodiments are described herein with reference to the treatment of diabetes, the skilled artisan will recognize that aspects of the present invention can advantageously be applied to many other fields.

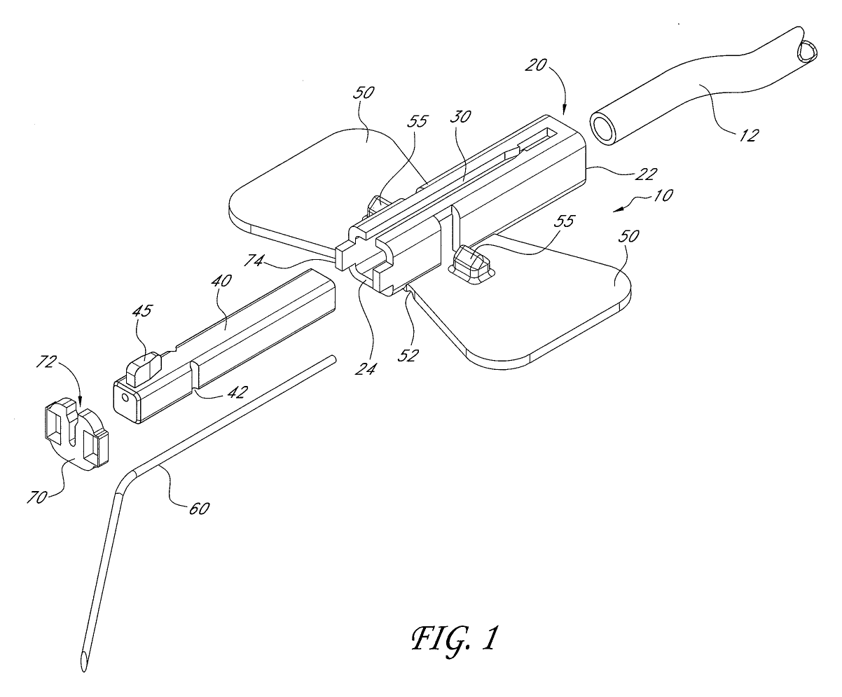

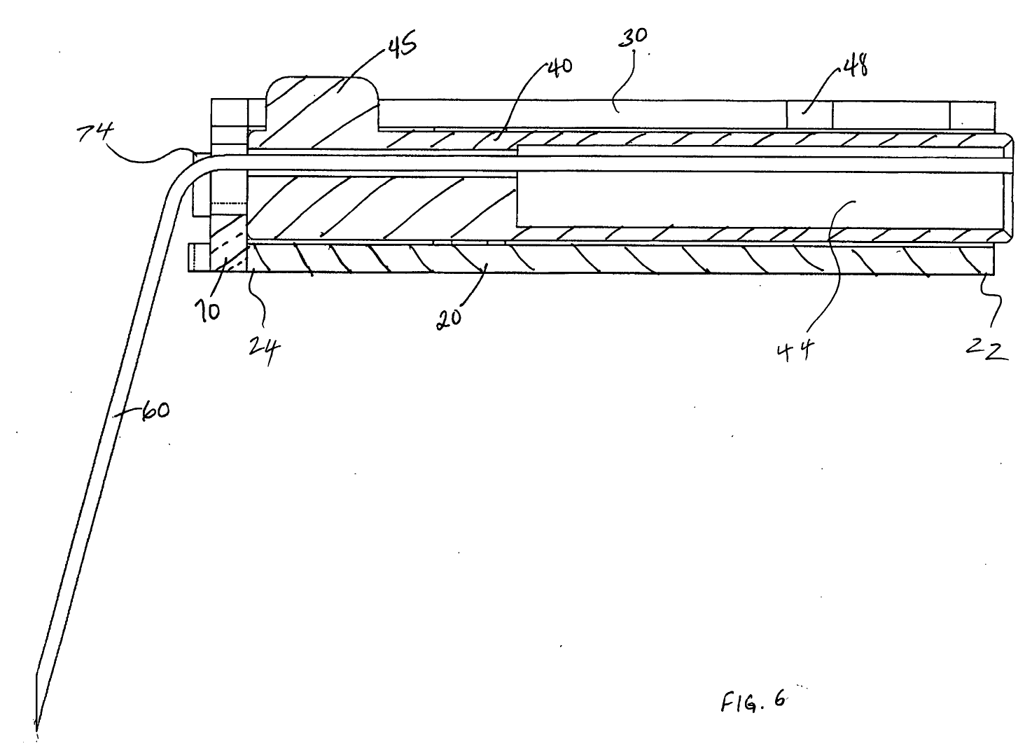

One embodiment of a percutaneous safety needle inserter 10 constructed in accordance with a preferred embodiment of the invention will now be described with reference to FIGS. 1-7. Embodiments of a needle inserter 10 generally comprise a housing 20 with a slider member 40 inserted therein. The slider member 40 generally includes a needle 60 extending from one end thereof, and an infusion tube 12 extending from an opposite end of the slider 40. The slider 40 is positioned within the housing 20 so as to be axially movable. A front plate 70 is secured to a downstream end 24 of the housing 20 and abuts a downstream end of the slider 40, thereby limiting the forward movement of the slider within the housing 20. Rearward movement of the slider is limited by a head member 45 extending upwards from the slider through a slot 30 in the housing 20.

FIG. 1 is an exploded perspective view of one embodiment of a safety needle inserter 10. As shown, the slider 40 is formed separately from the housing 20 so that the slider 40 may be slidably disposed within the housing 20. A portion of the needle 60 is threaded through a hole in the slider 40 and embedded within the slider 40. The sharp, distal end of the needle 60 is positioned toward the front end of the slider 40, with a bend in the needle 60 directing the needle tip downwards.

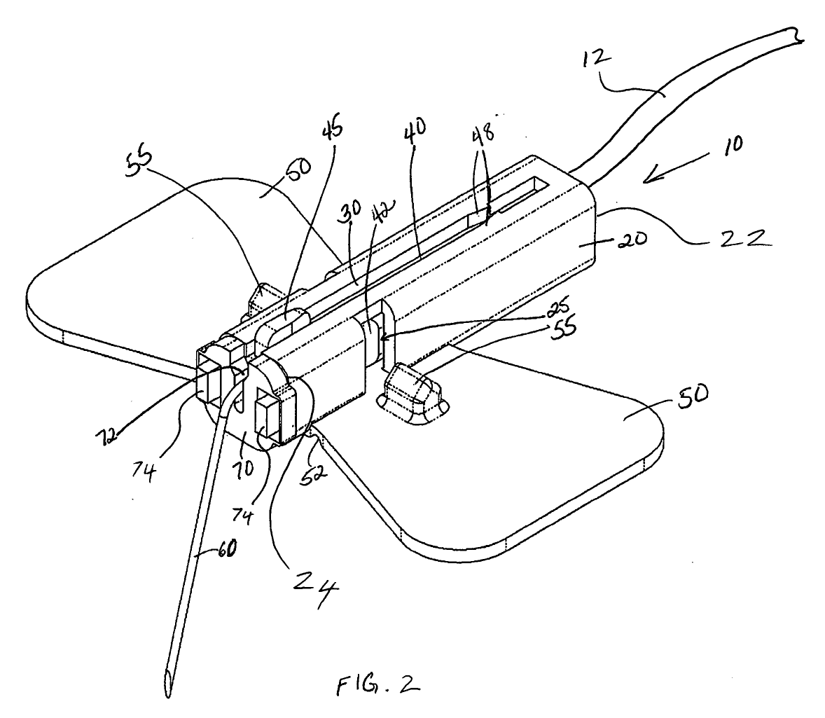

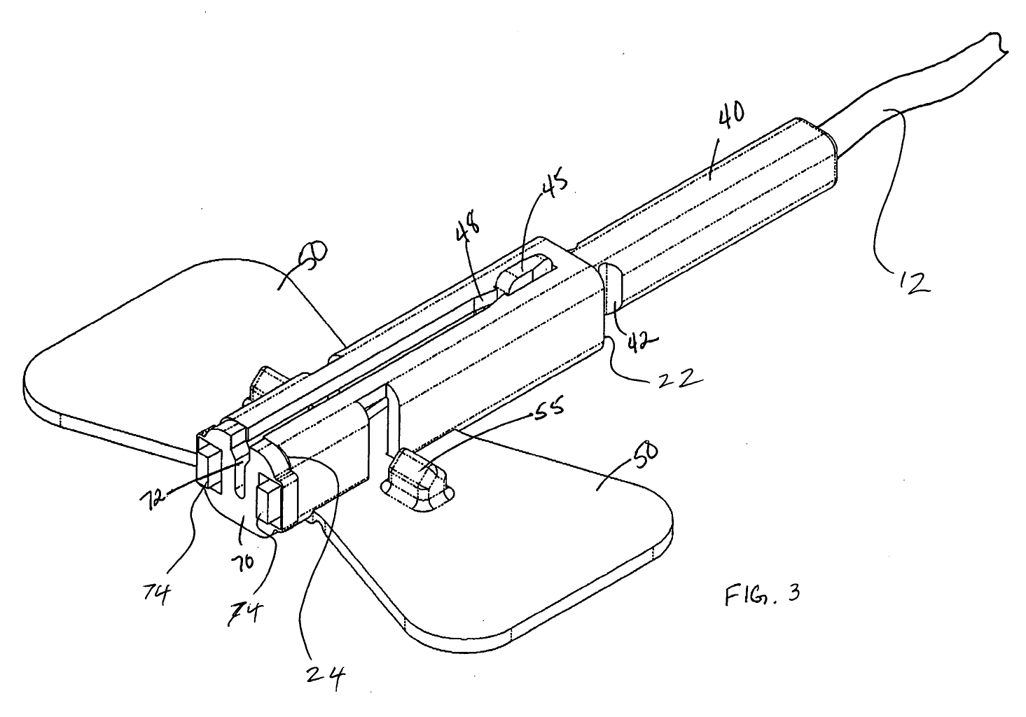

FIG. 2 is a perspective view of one embodiment of a percutaneous safety needle inserter 10 shown in a “use position,” while FIG. 3 is a perspective view of the needle inserter 10 in a “locked position.” The use position is the configuration of the needle inserter 10 in which it is capable of transmitting fluids to or from a patient. The “locked position” is the configuration of the needle inserter 10 in which the needle 60 is retracted within the housing 20 such that the sharp distal end of the needle 60 is no longer exposed, thereby guarding against injury.

The housing 20 is preferably hollow, elongated and has an upstream end 22 and a downstream end 24. The housing 20 comprises a longitudinal groove 30 on its top surface. The housing 20 preferably has a sufficient length such that a needle 60 may be at least partially retracted within the housing 20 in a locked position (as will be further described below). In a preferred embodiment, the housing 20 is fabricated from a single piece of injection molded plastic. In alternative embodiments, the housing (and other components) can be cast, machined or otherwise formed from any suitable material.

The longitudinal groove 30 preferably runs almost the entire length of the housing 20, and is typically generally rectangular in shape. In some embodiments, the groove 30 further comprises a pair of projections or barbs 48 towards the upstream end 22 of the housing 20. As will be discussed in more detail below, the barbs 48 are generally configured to retain the slider 40 and the needle 60 in a retracted or locked position in which the needle 60 does not protrude from the downstream end 24 of the housing 20.

The barbs 48 are preferably positioned toward the rear of the housing 40, as shown in FIGS. 2-4. The barbs 48 are preferably positioned far enough from the front plate 70 so that the sharp, distal tip of the needle 60 may be retracted within the housing 20 and the head portion 45 is locked in place past the barbs 48 (between the barbs 48 and the rear end of the longitudinal groove 30).

As shown in FIGS. 2-4, the barbs 48 are preferably angled toward each other and each barb 48 is a mirror image of the other barb 48, on opposite sides of the longitudinal axis of the housing 20. The barbs 48 are preferably angled toward each other such that the distance between the barbs 48 is smaller than the width of the longitudinal groove 30, and slightly smaller than a width of the head portion 45 of the slider 40. Thus, when the head portion 45 is slid in a rearward direction past the barbs 48 by the user, the head portion 45 cannot easily move past the barbs 48 in the forward direction.

The barbs 48 are configured such that the head portion 45 may be easily slid by a user part the barbs 48 in the rearward direction, but the head portion 45 cannot easily move past the barbs 48 in the forward direction. The barbs 48 are configured to prevent the slider 40 from moving in the forward direction to again expose the distal tip of the needle 60 beyond the front plate 70 of the housing 20 once the head portion 45 has been slid past the barbs 48 such that the needle is in the locked position. Preferably, the barbs 48 are formed of a material that provides enough flexibility to allow the slider 40 to pass through the narrow opening between the barbs 48. However, the barbs 48 should be stiff enough to prevent the slider 40 from moving forward past the barbs 48 to expose the distal tip of the needle 60 once it has been placed in the locked position. As understood by the skilled artisan, the barbs 48 may be in the form of any type of projections configured to allow the head portion 45 to move past them in the rearward direction, but not in the forward direction.

The housing 20 preferably has two wings 50 extending laterally from either side of the housing 20. Each wing 50 typically has a weakening groove 52 on its underside adjacent the housing to allow the wings 50 to be folded upwards.

Each wing 50 further includes a brace 55 that, in the upwardly folded position of the wing 50, passes through a respective opening 25 in the side of the housing 20 and engages a respective indent 42 in the slider 40. When the wings 50 are folded upwardly such that the braces 55 pass through the openings 25 and press against the indents 42 in the slider 40, the slider 40 is temporarily held in place and prevented from sliding relative to the housing. This temporary immobilization of the slider 40 stabilizes the slider 40 against axial movement during the injection stage. The needle 60 is also stabilized laterally by the front plate 70 through which the needle extends. The wings 50 are preferably formed of a moderately flexible material, such as polypropylene or any other suitable material. In some embodiments, the wings can further be provided with an adhesive layer on an underside thereof to further aid in securing the needle inserter to a patient's skin.

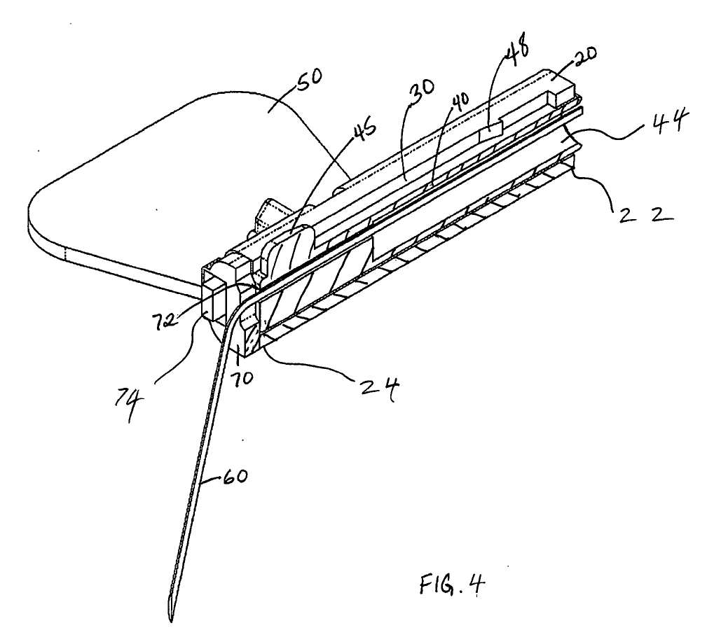

The elongated slider 40 can be clearly seen in FIG. 1, which shows the slider 40 withdrawn from the housing 20 in an exploded perspective view. With reference to FIG. 2, the slider 40 has a head portion 45 extending upwards through the longitudinal groove 30 and above the housing 20 such that the head portion 45 may be accessed by a user. As shown in FIGS. 4, 5 and 6, the head portion 45 protrudes vertically from the top surface of the slider 40. The head portion 45 is preferably formed toward the front end of the slider 40. In some embodiments, the head portion extends a sufficient distance above the housing 20 to allow a patient or clinician to engage the head 45 in order to move the slider 40 relative to the housing 20. In some embodiments, the head portion 45 can also be configured to engage the barbs 48 in order to retain the slider in the locked position.

The head portion 45 is preferably formed integrally with the slider 40, although in alternative embodiments, the head can be formed separately and attached by welding, adhesives or other suitable methods. The slider 40, including the head portion 45, is preferably formed of a fairly rigid material, such as hard plastic styrene, although other suitable materials acceptable for use in medical applications can also be used.

FIG. 1 also shows a front plate 70, which is attached to the downstream end 24 of the housing 20. In the illustrated embodiment, the front plate 70 is formed separately from the housing 20 in order to facilitate assembly of the needle inserter 10. Thus, the front plate 70 may be attached to the housing 20 after the slider 40 is slid into the housing 20. In the illustrated embodiment, the front plate 70 is engaged and retained on the housing 20 by barbed legs 74. In an alternative embodiment, the front plate 70 is formed integrally with the housing 20 of hard plastic styrene or other suitable material. The front plate also generally includes an aperture or slot 72 which is generally configured to retain the needle 60 against lateral movement relative to the housing 20. As best seen in FIGS. 4-7, the slot 72 preferably extends less than the height of the internal cavity of the housing 20. The skilled artisan will understand that although a slot is illustrated, the aperture 72 in the front plate 70 could alternatively be a circular hole, a J-shaped slot, an elliptical aperture or any other shape as desired. The skilled artisan will understand that the front plate 70 may be formed as part of the housing 20 or as a separate piece.

A first section of the needle 60 is embedded in the slider 40 and a second section (i.e. the sharp, distal end) extends outside of the downstream end of the slider 40. In some embodiments, the needle 60 can be threaded through a hole in the slider 40 after the slider 40 is molded. Alternatively the slider can be injection molded, cast or otherwise formed around the needle such that the needle is encapsulated in the slider. In general, the needle 60 is positioned within the slider 40 such that a portion of the upstream end of the needle 60 extends into a tubing cavity 44 at an upstream end of the slider 40. The infusion tubing 12 can then be secured within the tubing cavity 44 of the slider 40 by press fit, adhesives, sonic welds or any other suitable process.

In some embodiments, the distal end of the needle 60 (i.e. the portion extending from the slider) is preferably bent downwards from the longitudinal axis of the slider 40. In some embodiments, the needle is bent downwards from the longitudinal axis by anywhere from about 5° to about 90°. In some embodiments, the needle 60 is bent downwards by about 15° to 75°, and in one preferred embodiment by about 45°. In another embodiment, the needle 60 is bent downwards by about 90° relative to the slider longitudinal axis. In one preferred embodiment, the needle 20 is a flexible, subcutaneous safety needle of about 26 gauge or smaller, however the skilled artisan will recognize that larger or smaller needles can also be used as desired.

As shown in FIGS. 3, 5 and 7, the needle inserter 10 has a locked configuration to prevent inadvertent needle sticks and/or infection by the needle 60 after it is withdrawn from the patient. In the locked position, the sharp, distal tip of the needle 60 is fully retracted within the housing 20 to keep a used, and potentially contaminated, needle safely contained within the housing 20.

Another safety feature of the percutaneous needle inserter 10 is the bend in the needle 60, which is designed to keep the needle 60 within the housing 20 when it is in the locked position. The bend in the needle 60 creates a downward bias that causes the distal tip of the needle 60 to press downwardly against the bottom of the groove 30 in the housing 20, and away from the slot 72, after it is retracted to the locked position.

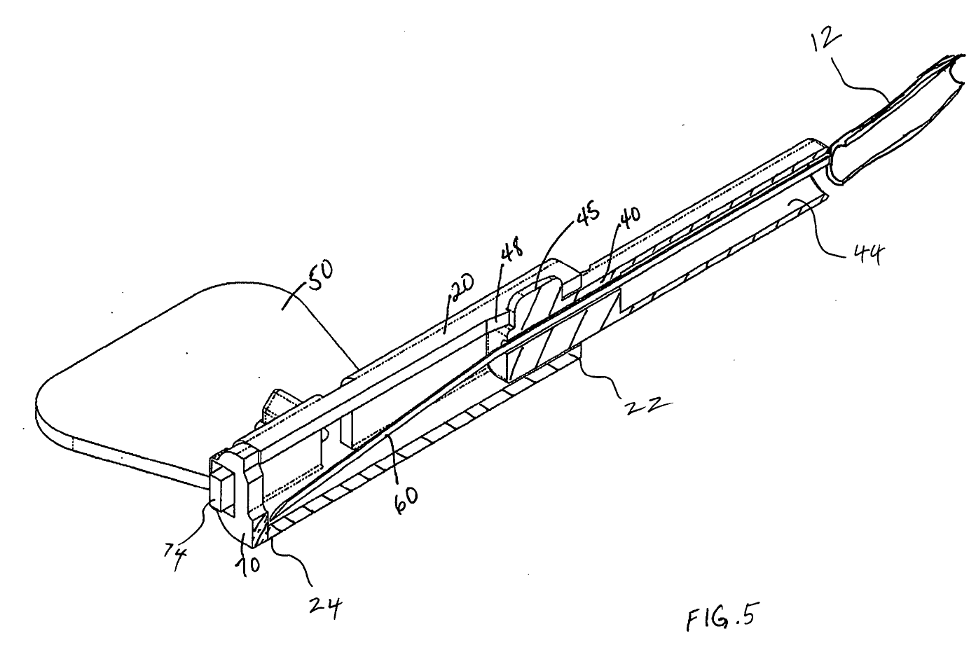

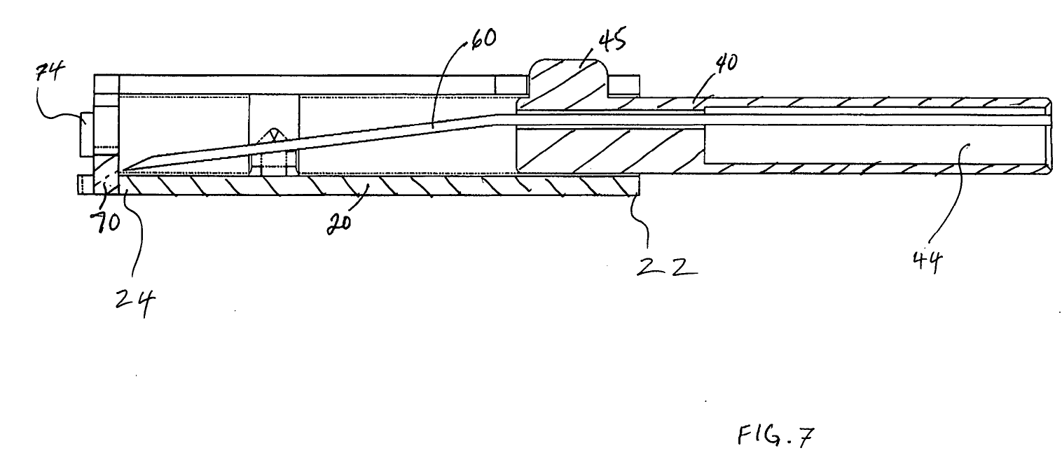

As best seen in FIGS. 5 and 7, the downward bias of the needle 60 causes the needle tip 60 to snap downwards within the housing 20 as it is drawn past the front plate 70. Because the slot 72 in the front plate 70 is preferably positioned a short distance above the bottom of the inside of the housing 20, as shown in FIG. 7, the distal tip of the needle 60 is blocked by the lower portion of the front plate 70 (below the slot 72) from extending outwardly after it is retracted. Thus, the needle 60 itself acts as a locking mechanism to retain the needle 60 within the housing 20 behind the front plate 70. As understood by the skilled artisan, the natural spring force of the needle 60 keeps the needle inserter 10 in the locked position.

The percutaneous safety needle inserter 10 also has a second locking mechanism, which comprises the head portion 45 of the slider 40 and the barbs 48 on the housing 20. Upon withdrawal of the needle 60 from the patient, the head portion 45 on the slider 40 is slid rearwardly relative to the housing 20 past the barbs 48, which engage the head portion 45 in the locked position. In one embodiment, this rearward movement of the slider head portion 45 causes the slider 40 to extend out of the rear 22 (i.e. upstream end) of the housing 20 and causes the needle 60 to retract to the locked position within the housing 20 as discussed above. As shown in FIGS. 3, 5 and 7, the entire needle 60, including the sharp, distal tip, is encased within the housing 20 in the locked position. Thus, the combination of the barbs 48 and the bend in the needle 60 ensures that a potentially contaminated used needle is kept within the housing 20 so that the danger of inadvertent needle pricks is substantially minimized. Preferably the head portion 45 or another mechanism does not permit the slider 40 from being removed from the housing 20, which would disadvantageously permit exposure to the needle 60.

With reference now to FIGS. 1-7, methods of using a needle inserter embodying features and advantages of the present invention will now be described. Typically, before injecting the needle 60 into a patient, each wing 50 is preferably bent upwards along the corresponding weakening groove 52 to fold upwardly against the housing 20. Folding the wings 50 up against the housing 20 allows the user to easily grasp the needle inserter in order to position the housing 20 accurately for insertion of the needle 60 in the patient. As discussed above, the upwardly folded wings 50 also advantageously retain the slider against axial motion relative to the housing as pressure is applied to the distal tip of the needle 60.

Once the needle 60 has been inserted into a patient's skin, the wings 50 can be folded down substantially parallel to the surface of the patient's skin such that the needle 60 and its attached wings 50 can be secured to the patient's skin by the use of an adhesive, such as adhesive tape. In some embodiments, each wing is individually taped to the patient's skin either by an adhesive layer on the wing itself, or by an additional piece of tape placed over the wing. If desired, a piece of medical tape can then be applied over the entire inserter in order to secure the slider 40, housing 20 and tubing 12 in place and to substantially prevent their movement relative to the wearer during use.

Once it becomes time to extract the needle 60 from a patient, the slider 40 can be moved in a rearward direction relative to the housing in order to simultaneously draw the needle out of the patient and into the housing 20. In one embodiment, the needle 60 is withdrawn by grasping and moving the head 45 of the slider 40 rearwards past the barbs 48, thereby locking the needle 60 within the housing 20 in the locked position. In an alternative embodiment, the needle 60 can be withdrawn by pulling the tubing 12 rearwardly relative to the housing 20 until the needle is fully retracted within the housing 20. Any adhesive holding the wings 50 can then be removed, and the inserter 10 can be safely discarded. In an alternative embodiment, the tape can be removed, and the needle 60 can be withdrawn from the patient before retracting the needle 60 into the housing 20.

Although this invention has been disclosed in the context of certain preferred embodiments and examples, it will be understood by those skilled in the art that the present invention extends beyond the specifically disclosed embodiments to other alternative embodiments and/or uses of the invention and obvious modification thereof Thus, it is intended that the scope of the present invention herein disclosed should not be limited by the particular disclosed embodiments described above, but should be determined only by a fair reading of the claims that follow.

1. An infusion device comprising:
an elongate housing member having a cavity extending therethrough and having a front plate secured to a first end thereof;
a slider member positioned within the cavity so as to be axially slidable therein, the slider member having a needle extending from a first end thereof, said needle extending through an aperture in the front plate when the slider is in a position near the front plate;
wherein the slider is configured to slide in a direction away from the front plate to a position in which the needle is retained within the housing member, and said needle being non-straight and substantially restrained from forward movement by the front plate when the slider is in a position away from the front plate; and
wherein the infusion device is configured to permit fluid flow through the housing member and slider member.

2. The device of claim 1, further comprising a locking mechanism configured to selectively prevent the slider from moving axially relative to the housing.

3. The device of claim 1, wherein the needle is bent at an angle of between about 5° and about 90° relative to a longitudinal axis of the slider member.

4. The device of claim 1, further comprising a longitudinal groove in an upper surface of the housing and a head portion attached to the slider and extending upwards through the groove.

5. The device of claim 4, further comprising projections extending inwardly from walls of the longitudinal groove.

6. The device of claim 5, wherein the projections are configured to allow the head portion to pass between them in one direction but not in an opposite direction.

7. The safety needle inserter of claim 6, wherein the projections are barbs.

8. The device of claim 1, wherein the needle is a flexible, subcutaneous needle of about 26 gauge or smaller.

9. The device of claim 1, further comprising foldable wings projecting laterally from the housing.

10. The safety needle inserter of claim 9, wherein each of the wings has a protrusion, the housing has apertures, and the slider has indents, wherein each of the apertures and indents is configured to receive a corresponding protrusion when each of the wings is folded up against sides of the housing.

11. The device of claim 1, wherein the housing comprises a bottom interior surface and wherein the aperture in the front plate is positioned above the bottom interior surface.

12. The device of claim 11, wherein at least a portion of the needle is biased downward toward the bottom interior surface.

13. The device of claim 1, further comprising a section of infusion tubing secured to a second end thereof.

14. A method of preventing injury from an infusion needle, the method comprising:
locating an infusion set comprising a slider member having a needle extending from a front end thereof into a patient, the slider member being slidably positioned within a housing member, the infusion set configured to permit fluid flow through the housing member and slider member;
removing the needle from the patient; and
holding the housing member stationary while moving the slider member rearwards until a sharp tip of the needle is completely retracted within the housing, the needle being non-straight and substantially restrained from forward movement by a front plate of the housing member when the sliding member is in a position away from the front plate.

15. The method of claim 14, wherein removing the needle from the patient is performed simultaneously with moving the slider member relative to the housing.

16. The method of claim 14, further comprising discarding the needle, housing and slider.

17. The method of claim 14, wherein moving the slider member comprises grasping a head extending upwards through a slot in the housing member.

18. The method of claim 14, wherein moving the slider member comprises pulling rearwardly on a section of tubing secured to the slider.

19. The device of claim 1, wherein the needle is a flexible, subcutaneous needle of about 27 gauge or smaller.
